(19) United States
(12) Patent Application Publication
(10) Pub. No.: US 2024/0161872 A1
(43) Pub. Date: May 16, 2024

(54) METHOD AND SYSTEM FOR OPTIMAL VACCINE DESIGN

(71) Applicant: NEC Corporation, Tokyo (JP)

(72) Inventors: Brandon MALONE, Heidelberg (DE); Jun CHENG, Heidelberg (DE)

(73) Assignee: NEC Corporation, Tokyo (JP)

(21) Appl. No.: 18/424,042

(22) Filed: Jan. 26, 2024

Related U.S. Application Data
(63) Continuation of application No. 17/788,304, filed on Jun. 23, 2022.

(30) Foreign Application Priority Data
Apr. 20, 2020 (EP) 20170475.6

(51) Int. Cl. G16B 30/20; G16B 5/20; G16B 20/40; G16B 40/20

(52) U.S. Cl. CPC G16B 30/20; G16B 5/20; G16B 20/40; G16B 40/20

(57) ABSTRACT

A computer-implemented method of selecting one or more amino acid sequences for inclusion in a vaccine from a set of predicted immunogenic candidate amino acid sequences includes identifying an immune profile response value for each candidate amino acid sequence with respect to each one of a plurality of sample components of an immune profile. The immune profile response value represents whether the respective candidate amino acid sequence results in an immune response for the sample components of the immune profile. A plurality of immune profiles are retrieved for a population. A plurality of representative immune profiles are generated for the population. The representative immune profiles overlap with the sample components of the immune profiles. The one or more amino acid sequences for inclusion in the vaccine that minimises a likelihood of no immune response for each representative immune profile, based on the immune profile response values, are selected.

### CROSS REFERENCE TO RELATED APPLICATIONS

This application is a continuation application of U.S. application Ser. No. 17/788,304, filed on Jun. 23, 2022, which is a U.S. National Phase application under 35 U.S.C. § 371 of International Application No. PCT/EP2020/068109, filed on Jun. 26, 2020, and claims benefit to European Patent Application No. EP 20170475.6, filed on Apr. 20, 2020. The International Application was published in English on Oct. 28, 2021 as WO 2021/213687 A1 under PCT Article 21(2).

### FIELD

The present invention relates to a method and system for vaccine design.

### BACKGROUND

Epitope-based vaccines (EVs) make use of short antigen-derived peptides corresponding to immune epitopes, which are administered to trigger a protective humoral and/or cellular immune response. EVs potentially allow for precise control over the immune response activation by focusing on the most relevant—immunogenic and conserved—antigen regions. Experimental screening of large sets of peptides is time-consuming and costly; therefore, in silico methods that facilitate T-cell epitope mapping of protein antigens are paramount for EV development. The prediction of T-cell epitopes focuses on the peptide presentation process by proteins encoded by the major histocompatibility complex (MHC). Because different MHCs have different specificities and T-cell epitope repertoires, individuals are likely to respond to a different set of peptides from a given pathogen in genetically heterogeneous human populations. In addition, protective immune responses are only expected if T-cell epitopes are restricted by MHC proteins expressed at high frequencies in the target population. Therefore, without careful consideration of the specificity and prevalence of the MHC proteins, EVs could fail to adequately cover the target population.

Vaccine design in the context of genetically heterogeneous human populations faces two major problems: first, individuals displaying a different set of alleles, with potentially different binding specificities, are likely to react with a different set of peptides from a given pathogen; and second, alleles are expressed at dramatically different frequencies in different ethnicities.

Computational tools can be valuable in dealing with these issues in vaccine design. Available computational methods for T-cell epitope vaccine design mostly focus on the stage of epitope prediction of peptide binding to MHCs. A lesser number of tools and algorithms have been developed to guide the selection of putative epitopes, either by maximizing coverage in the target population and/or in terms of pathogen diversity, and to optimize the design of polypeptide vaccine constructs.

Current state of the art approaches to epitope-based vaccine design, and specifically the challenge of selecting putative epitopes, are broadly classified as HLA supertype-based and allele-based (Oyarzun, P. & Kobe, B. Computer-aided design of T-cell epitope-based vaccines: addressing population coverage. International Journal of Immunogenetics, 2015, 42, 313-321).

Supertype-based methods are known to perform poorly for populations with diverse HLA backgrounds by favouring only the most common HLA alleles (Schubert, B.; Lund, O. & Nielsen, M. Evaluation of peptide selection approaches for epitope-based vaccine design. Tissue Antigens, 2013, 82, 243-251).

Current state-of-the-art, allele-based approaches do not consider individual citizens when selecting elements for inclusion in the vaccine; rather, they aim to maximize the average likelihood of response for all individuals. This is problematic because the proposed approaches will focus on eliciting the strongest (or most likely) responses possible rather than ensure each citizen is protected by the vaccine (Vider-Shalit, T.; Raffaeli, S. & Louzoun, Y. Virus-epitope vaccine design: Informatic matching the HLA-I polymorphism to the virus genome. Molecular Immunology, 2007, 44, 1253-1261; Toussaint, N. C.; Dönnes, P. & Kohlbacher, O. A Mathematical Framework for the Selection of an Optimal Set of Peptides for Epitope-Based Vaccines. PLOS Computational Biology, 2008, 4, e1000246; Lundegaard, C.; Buggert, M.; Karlsson, A. C.; Lund, O.; Perez, C. & Nielsen, M. PopCover: A Method for Selecting of Peptides with Optimal Population and Pathogen Coverage. Proceedings of the 1\hat{ }st ACM International Conference on Bioinformatics and Computational Biology, 2010)

Other known approaches use a graph-based approach to design epitope vaccines, but none of these approaches have been shown to produce optimal vaccine designs (Theiler, J. & Korber, B. Graph-based optimization of epitope coverage for vaccine antigen design. Statistics in Medicine, 2018, 37, 181-194).

### SUMMARY

In an embodiment, the present invention provides a computer-implemented method of selecting one or more amino acid sequences for inclusion in a vaccine from a set of predicted immunogenic candidate amino acid sequences. The method includes identifying an immune profile response value for each candidate amino acid sequence with respect to each one of a plurality of sample components of an immune profile. The immune profile response value represents whether the respective candidate amino acid sequence results in an immune response for the sample components of the immune profile. A plurality of immune profiles are retrieved for a population. A plurality of representative immune profiles are generated for the population. The representative immune profiles overlap with the sample components of the immune profiles. The one or more amino acid sequences for inclusion in the vaccine that minimises a likelihood of no immune response for each representative immune profile, based on the immune profile response values, are selected.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic view of an exemplary tripartite graph according to an embodiment of the invention;

FIG. 2 shows a high-level flowchart of an approach according to an embodiment of the invention;

FIG. 3 shows an alternative schematic view of an exemplary tripartite graph according to an embodiment of the invention;

FIG. 4 shows an example output; and

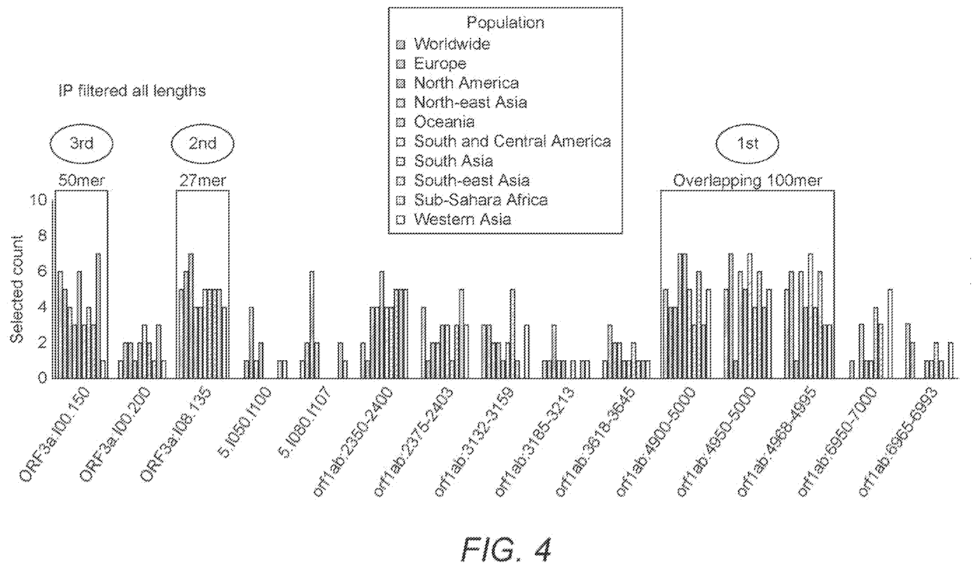

FIG. 5 shows a method according to an embodiment of the present invention.

### DETAILED DESCRIPTION

Embodiments of the invention provide a method and system for selecting a set of candidate elements for inclusion in a vaccine such that the likelihood that every member of a population has a positive response to the vaccine is maximized. Embodiments of the invention improve on existing methods for selecting candidate elements for inclusion in a vaccine.

According to an embodiment of the present invention, there is provided a computer-implemented method of selecting one or more amino acid sequences for inclusion in a vaccine from a set of predicted immunogenic candidate amino acid sequences, the method comprising: identifying an immune profile response value for each candidate amino acid sequence in respect of each one of a plurality of sample components of an immune profile, wherein the immune profile response value represents whether the candidate amino acid sequence results in an immune response for the sample component of an immune profile; retrieving a plurality of immune profiles for a population; generating a plurality of representative immune profiles for the population, wherein the representative immune profiles overlap with the sample components of an immune profiles; and, selecting the one or more amino acid sequences for inclusion in the vaccine that minimises a likelihood of no immune response for each representative immune profile, based on the immune profile response values.

Advantageously the proposed approach explicitly accounts for and optimizes with respect to a wide variety of components the make up an immune profile, in contrast to the approaches of the state of the art, and maximises the chances of a vaccine being a success across a given population. Where the population is representative of the global population, the approach can be considered to lead toward an optimal, universal vaccine, that is, that the chances of an immune response being caused by the combination of vaccine elements included in the vaccine is maximised. For example, where the sample components are a plurality of sample HLA alleles, the proposed approach explicitly accounts for and is optimized with respect to all alleles.

In sum, the method of the above embodiment of the invention formulates a vaccine design with respect to a specific population as an optimization problem in which the goal is to maximize the likelihood of response of each citizen.

The present technique may be thought of as an allele-based approach; however, unlike the methodology of the art, the current approach considers individual citizens rather than looking at the most frequently occurring alleles in a population and seeking to provide an average across that set. We note that in the art population coverage describes the fraction of a population for which the epitope based vaccine is theoretically effective.

The predicted immunogenic candidate amino acid sequences may be short or long peptide sequences, where a long peptide sequence may include multiple short peptide sequences. The set of predicted immunogenic candidate amino acid sequences are typically retrieved from a prediction engine which computes some sort of a score that a peptide will result in some immune response (e.g., binding, presentation, cytokine release, etc.). Examples of publically available databases and tools that may be used for such predictions include the Immune Epitope Database (IEDB) (https://www.iedb.org/), the NetMHC prediction tool (http://www.cbs.dtu.dk/services/NetMHC/) and the NetChop prediction tool (http://www.cbs.dtu.dk/services/NetChop/). Other techniques are disclosed in WO2020/070307 and WO2017/186959.

The score from the prediction engine associated with each sequence may be used to identify the immune response value. Alternatively the immune response value may be retrieved from a database populated using data in previous literature, for example, by extracting univariate response statistics.

The one or more predicted candidate amino acid sequences may be of a fixed length or of variable lengths. For example, when considering MHC Class I HLA alleles, epitope lengths of 8, 9, 10, 11 and 12 amino acids may be candidates and when considering MHC Class II HLA alleles, each epitope is typically 15 amino acids in length. Alternatively, the candidate amino acid sequences may be groups of sequences. Example, candidate amino acid sequences include: (1) short peptide sequences, such as 9-mer amino acid sequences; (2) long peptide sequences, such as 27-mer amino acid sequence which may be based on a short peptide sequence and include flanking regions; (3) longer amino acid sequences which may include multiple short peptide sequences as well as the intervening, naturally-occurring sequence; and (4) entire protein sequences.

The step of selecting the one or more amino acid sequences for inclusion in the vaccine may also be based on a correspondence between the sample components of an immune profile and the components of the immune profile present in the respective representative immune profiles.

In certain embodiments the immune profile may comprise one or more selected from a group comprising: a set of HLA alleles; presence (or absence) of tumor infiltrating lymphocytes; presence (or absence) of immune checkpoint markers, such as PD1, PD-L1, or CTLA4; presence (or absence) of hypoxia markers, such as HIF-1a or BNIP3; presence (or absence) of chemokine receptors such as CXCR4, CXCR3, and CX3CR1; and, previous infection by human papillomavirus. Each of these features has been shown to contribute, positively or negatively, to the immune response of a particular epitope, or candidate vaccine element. Thus the immune response value associated with each candidate amino acid sequence may represent the contribution of how likely that candidate sequence is to produce an immune response with the particular variables in question.

In specific embodiments the sample components of an immune profile comprise a sample HLA allele, such that the immune profile response value comprises an HLA allele immune response value for each candidate amino acid sequence in respect of each one of a plurality of sample HLA alleles. The immune profiles for a population may comprise a plurality of HLA genotypes for a population. The step of generating a plurality of representative immune profiles may comprise generating a plurality of representative sets of HLA alleles for the population. The HLA alleles of the representative sets may overlap with the sample HLA alleles.

The sample HLA alleles of the immune profile may be a set of most frequently occurring alleles in a population or all alleles of a population. A degree of overlap between the sample HLA alleles and the representative immune profiles may include: (1) that all sample HLA alleles occur within at least one representative immune profile; and/or (2) that all HLA alleles of the representative immune profiles occur within the sample HLA alleles. Preferably at least one allele for each representative immune profile needs to be in the set of sample HLA alleles. Preferably each of the sample HLA alleles should be present in at least one of the representative sets. Similar variations in degrees of overlap are contemplated between the components of the immune profile and the representative immune profiles.

In implementations, the candidate amino acid sequences are vaccine elements and each representative set is a simulated citizen of a given population.

The method may further comprise retrieving a set of predicted immunogenic candidate amino acid sequences. The retrieval may be from a local memory, database or remote data repository.

In preferred embodiments, the step of generating comprises: (i) creating a first distribution over the plurality of immune profiles; and, (ii) sampling the first distribution to create the plurality of representative immune profiles. In examples, the immune profiles may comprise HLA genotypes.

More preferably, the first distribution is a distribution over the plurality of immune profiles for each region of the population.

Each region may be a population group having an ethnic population group (e.g. Caucasian, Africa, Asian) or a geographical population group (e.g. Lombardy, Wuhan).

Even more preferably, the first distribution is a posterior distribution over genotypes in each region based on a prior distribution and observed genotypes from the plurality of immune profiles in each region of the population.

In certain specific implementations, the first distribution is a symmetric Dirichlet distribution, wherein the method further comprises the step of collecting all genotypes observed at least once across all regions, and wherein the step of sampling comprises sampling a desired number of genotypes from each region based on counts of each genotype in the sample. An alternative to a Dirichlet may be a multivariate Gaussian followed by a logistic function transformation.

Advantageously, the present approach considers insufficiencies of the input data and is able to properly account for limitations in the data samples which were used to populate the input database. To do so, the method preferably comprises simulating a digital population based on the retrieved plurality of immune profiles for the population, wherein the step of creating a first distribution is based on the simulated population such that the step of sampling is performed on the simulated population.

Such simulation may be thought of as creating a “digital twin” of the citizens in the population present in the database, where the “digital twin” is an immune profile and may for example include a set of HLA alleles and other indicators of immune response, such as previous infection by human papillomavirus. In this way, the methodology adopts a “digital twin” framework in which synthetic populations are simulated, and an optimal selection of vaccine elements is made with respect to that simulation.

If, for example, the input database comprises 400 people from a particular region then it may be advisable to augment the available data. The proposed statistical models can create or simulate people matching actual people in the region to create an increased number of citizens, such as 10,000.

The proposed models include a degree of variance. By creating a posterior distribution over the genotypes, the variation may be proportional to the amount of genotypes in the database.

Specifically, the step of simulating a digital population comprises: defining a population size; and, creating a second distribution over the regions.

In a specific implementation, the second distribution is a Dirichlet distribution. A contemplated alternative to a Dirichlet is a multivariate Gaussian followed by a logistic function transformation.

The proposed models emphasise rare genotypes to ensure that there is maximum coverage of the population. This is in contrast to existing approaches which look at the most frequently occurring alleles in order to try to maximise the coverage of the vaccine. These approaches inherently ignore rare genotypes and hence are unsuitable for a universal vaccine as, although they will be useful for the majority of the population, the vaccine provides no benefit for the minority. Moreover, by looking at frequently occurring alleles, the approaches are biased towards the inherent deficiencies of the input database. Where, for example, there is poor data for a region, frequently occurring alleles in that region will not be emphasised creating an inherent bias in the chosen vaccine elements towards regions with good data coverage in the input database.

Typically, the representative immune profiles are generated such the representative immune profiles maximise coverage of combinations of immune profiles in the population.

The step of selecting is typically performed so as to choose amino acid sequences which provide the best possible vaccine. In preferred implementations, the step of selecting comprises applying a mathematical optimisation algorithm to minimise a maximum likelihood of no immune response for each representative immune profile.

In effect, the approach aims to calculate the likelihood of no response for a given representative immune profile and a given set of amino acid sequences. This may be thought of as a sum of the immune response values for the sample components of an immune profile corresponding to the components in the representative immune profile.

The mathematical optimisation algorithm may be constrained by one or more predetermined thresholds. In embodiments, the amino acid sequences may be selected based on a particular vaccine delivery platform.

Typical algorithms may struggle with such computational complexity and so to provide efficiencies and improvements, the method may be configured to provide one or more surrogate variables for the mathematical optimisation algorithm. The surrogate variables may comprise a log likelihood of no response for a representative set. In a specific preferred implementation, variables of the mathematical optimisation algorithm comprise: (a) a binary indicator variable for each candidate amino acid sequence which indicates whether the candidate amino acid is included in a vaccine; (b) a continuous variable for each representative immune profile which gives a log likelihood of no immune response; (c) a continuous variable for each sample component which gives a log likelihood of no response; and, (d) a continuous variable which gives a maximum log likelihood that any representative immune profile does not respond to the selected one or more amino acid sequences, wherein the mathematical optimisation algorithm minimises the continuous variable which gives a maximum log likelihood that any representative immune profile does not respond to the selected one or more amino acid sequences.

Accordingly, in a certain embodiments, the immune profile may comprise a set of HLA alleles and the sample components of an immune profile may comprise sample HLA alleles. In these embodiments, optionally the variables of the mathematical optimisation algorithm may comprise: (a) a binary indicator variable for each candidate amino acid sequence which indicates whether the candidate amino acid is included in a vaccine; (b) a continuous variable for each representative immune profile which gives a log likelihood of no immune response; (c) a continuous variable for each sample component of an immune profile which gives a log likelihood of no response; and, (d) a continuous variable which gives a maximum log likelihood that any representative immune profile does not respond to the selected one or more amino acid sequences, wherein the mathematical optimisation algorithm minimises the continuous variable which gives a maximum log likelihood that any representative immune profile does not respond to the selected one or more amino acid sequences.

An objective of the mathematical optimisation algorithm is to minimize variable (d). In embodiments, the setting of the binary variables corresponds to the optimal choice of amino acid sequences for the given population. Advantageously the mathematical optimisation algorithm is a mixed integer linear program.

In this way the optimisation can take advantages of the benefit of such programming since the decisions are binary, i.e. whether or not to include an amino acid sequence in the vaccine.

Choosing amino acid sequences for inclusion in a vaccine is not an unlimited exercise and selection is preferably constrained in some way. Preferably, the method further comprises: assigning a cost to each candidate amino acid sequence, wherein the step of selecting is constrained based on the cost assigned to each candidate amino acid sequence, such that the selected one or more amino acid sequences have a total cost below a predetermined threshold budget.

Accordingly, an amount of amino acid sequences to be included in the vaccine can be selected based on the practical realities of the chosen vaccine platform and the vaccine delivery method. Additionally, or alternatively, the step of selecting is constrained based on a maximum amount of amino acid sequences allowed in a vaccine delivery platform.

Optionally, this may be performed by assigning a cost of 1 to each amino acid sequence and a budget according to the number of amino acid sequences that can be included in the vaccine.

In addition to being considered an allele-based approach, a proposed embodiment may also be thought of as a graph-based approach in which, the method further comprises creating a tripartite graph, wherein: a first set of nodes corresponds to the candidate amino acid sequences; a second set of nodes corresponds to the sample components of an immune profile; and, a third set of nodes corresponds to the representative immune profiles for the population, and wherein: weights of edges between the first set of nodes and the second set of nodes are the immune response values; and, weights of edges between the second set of nodes and the third set of nodes represent correspondence between the sample components and each representative immune profile.

Thus the implementation may be thought of as a network flow problem through the graph in which a minimax problem is handled with the goal of choosing a set of vaccine elements which minimize the log likelihood of no response for each hypothetical citizen. Conventional graph-based approaches do not consider the population HLA background.

In preferred embodiments the immune response value is a log likelihood value based on amino acid sub-sequences of the candidate amino acid sequence.

The vaccine design approach is applicable for any approach which assigns a value for a log likelihood. Most short peptide prediction engines compute some sort of a score that a peptide will result in some immune response (e.g., binding, presentation, cytokine release, etc.), and this score generally takes into account a specific HLA allele. In some cases, this is already a probability, and in others, it can be converted into a probability using a transformation function, such as a logistic function. Additionally, the step of identifying comprises selecting a best likelihood value as the immune response value from a likelihood value for each amino-acid subsequence.

Thus, where the candidate amino acid sequences comprise multiple peptide sequences, the likelihood values can be determined based on a score for each short peptide sequence that goes into a long or longer peptide sequence.

In particularly preferred embodiments the one or more candidate amino acid sequences are comprised in one or more proteins of a coronavirus, preferably the SARS-CoV-2 virus.

In this way the approach is suitable for providing a universal, optimised vaccine design across a population of interest for the SARS-CoV-2 virus. In examples, the one or more candidate amino acid sequences may be one or more of the Spike (S) protein, Nucleoprotein (N), Membrane (M) protein and Envelope (E) protein of a virus, as well as open reading frames, such as orflab. Thus, an embodiment of the method of the present invention may be applied to an entire virus proteome. This is particularly beneficial for the identification of candidate elements for vaccine design.

The method may further comprise synthesising one or more selected amino acid sequences.

The method may further comprise encoding the one or more selected amino acid sequences into a corresponding DNA or RNA sequence. Further, the method may comprise incorporating the DNA or RNA sequence into a genome of a bacterial or viral delivery system to create a vaccine.

Thus, according to an embodiment of the invention there is provided a method of creating a vaccine, comprising: selecting one or more amino acid sequences for inclusion in a vaccine from a set of predicted immunogenic candidate amino acid sequences by a method according to any of the above aspects; and synthesising the one or more amino acid sequences or encoding the one or more amino acid sequences into a corresponding DNA or RNA sequence and/or incorporating the DNA or RNA sequence into a genome of a bacterial or viral delivery system to create a vaccine.

According to a further embodiment of the invention there may be provided a computer-implemented method of selecting one or more amino acid sequences for inclusion in a vaccine from a set of predicted immunogenic candidate amino acid sequences, the method comprising: retrieving a set of predicted immunogenic candidate amino acid sequences; identifying an HLA allele immune response value for each candidate amino acid sequence in respect of each one of a plurality of sample HLA alleles, wherein the HLA allele immune response value represents if the candidate amino acid sequence results in an immune response for the sample HLA allele; retrieving a plurality HLA genotypes for a population; generating a plurality of representative sets of HLA alleles for the population, wherein the HLA alleles of the representative sets overlap with the sample HLA alleles; selecting the one or more amino acid sequences for inclusion in the vaccine that minimises a likelihood of no immune response for each representative set of HLA alleles, based on the HLA allele immune response values and a correspondence between the sample HLA alleles and the HLA alleles present in the respective representative set of HLA alleles.

In accordance with a further embodiment of the invention there is provided a system for selecting one or more amino acid sequences for inclusion in a vaccine from a set of predicted immunogenic candidate amino acid sequences, the system comprising at least one processor in communication with at least one memory device, the at least one memory device having stored thereon instructions for causing the at least one processor to perform a method according to any of the above aspects.

In accordance with a further embodiment of the invention there is provided a computer readable medium having computer executable instructions stored thereon for implementing the method of any of the above aspects.

According to certain embodiments described herein there is proposed a method and system for selecting a small set of candidate elements for inclusion in a vaccine such that the likelihood that every member of a population has a positive response to the vaccine is maximized. Specifically, there is a focus on epitope-based vaccines. A “digital twin” framework is adopted in which synthetic populations are simulated, and an optimal selection of vaccine elements is made with respect to that simulation.

In this document, there is proposed a method and a system to design a vaccine which is effective against SARS-CoV-2 and other infections. There is a focus on epitope-based vaccines, in which a vaccine consists of a set of epitopes, or short amino acid sequences (Patronov, A. & Doytchinova, I. T-cell epitope vaccine design by immunoinformatics. Open Biology, 2013, 3, 120139. and Caoili, S. E. C. Benchmarking B-Cell Epitope Prediction for the Design of Peptide-Based Vaccines: Problems and Prospects. Journal of Biomedicine and Biotechnology, 2010). In particular, the present system preferably selects from among a set of candidate elements to include in a vaccine by simulating a population of “digital twin” citizens; in this context, a digital twin may comprise the human leukocyte antigen (HLA) profile of a citizen. The HLA profile is a key determinant in the immune response that a particular citizen can mount in response to infection (Shiina, T.; Hosomichi, K.; Inoko, H. & Kulski, J. K. The HLA genomic loci map: expression, interaction, diversity and disease. Journal of Human Genetics, 2009, 54, 15-39), and it is also an important factor for determining whether a vaccine is effective in establishing immunity for the specific individual.

The method is also applicable to considering immune profiles of a population where the digital twin comprises an HLA profile and/or further aspects that may contribute to the immune response for a particular vaccine. For example, components of such an immune profile may comprise presence (or absence) of tumor infiltrating lymphocytes; presence (or absence) of immune checkpoint markers, such as PD1, PD-L1, or CTLA4; presence (or absence) of hypoxia markers, such as HIF-1a or BNIP3; presence (or absence) of chemokine receptors such as CXCR4, CXCR3, and CX3CR1; and, previous infection by human papillomavirus.

The following sets out a specific example of the selection of candidate elements for a vaccine. In the proposed implementation set out below, note that any references indicated herein are incorporated by reference. Based on the HLA profile of the citizens in a population, it is proposed to select the set of vaccine elements to include in the vaccine (while respecting a budget of what can be included in the vaccine).

A population may be considered as a set C of “digital twin” citizens c, and a vaccine as a set V of vaccine elements v. The likelihood that all citizens have a positive response to a vaccine is denoted here as P(R=+|C, V). The goal is to design a vaccine, that is, select a set of vaccine elements, to maximize this probability:

$${max}_{V} P ( R = + \vert V , C )$$

In this setting, maximizing the probability of positive response is the same as minimizing the probability of no response. Thus, one can approach vaccine design by minimizing the probability of no response for the citizen who has the highest probability of no response P(R=−|V, c):

$${max}_{V} P ( R = + \vert V , C ) := {min}_{V} {max}_{{c}_{j} ∈ C} \{ P ( R = - \vert V , c ) \}$$

A vaccine may be considered to cause a response if at least one of its elements causes a positive response. That is, the probability of no response is the joint likelihood that all elements fail. For a particular citizen cⱼ, this probability is given as follows.

$$P ( R = - \vert V , c ) = {\prod }_{{v}_{i} ∈ V} P ( R = - \vert v , {c}_{j} , V )$$

We note that the conditioning set of the likelihood includes V.

The original optimization problem can then be expressed as:

$${min}_{V} {max}_{{c}_{j} ∈ C} {\prod }_{{v}_{i} ∈ V} P ( R = - \vert {v}_{i} , {c}_{j} , V )$$

Since the logarithm function is monotonic, the value of V which minimizes the logarithm of the function also minimizes the original function.

$${min}_{V} {max}_{{c}_{j} ∈ C} {\prod }_{{v}_{i} ∈ V} log P ( R = - \vert {v}_{i} , {c}_{j} , V )$$

Further, each citizen may be considered as an immune profile. The immune profile may comprise a set of HLA alleles and/or further components, as set out below. It can be assumed that each vaccine element vᵢ may result in a response on each allele or component of the immune profile independently. The alleles or components can be referred to, for citizen cⱼ, as A(cⱼ). Thus, the final objective is as follows.

$${min}_{V} {max}_{{c}_{j} ∈ C} {\prod }_{{v}_{i} ∈ V} log P ( R = - \vert {v}_{i} , k , V )$$

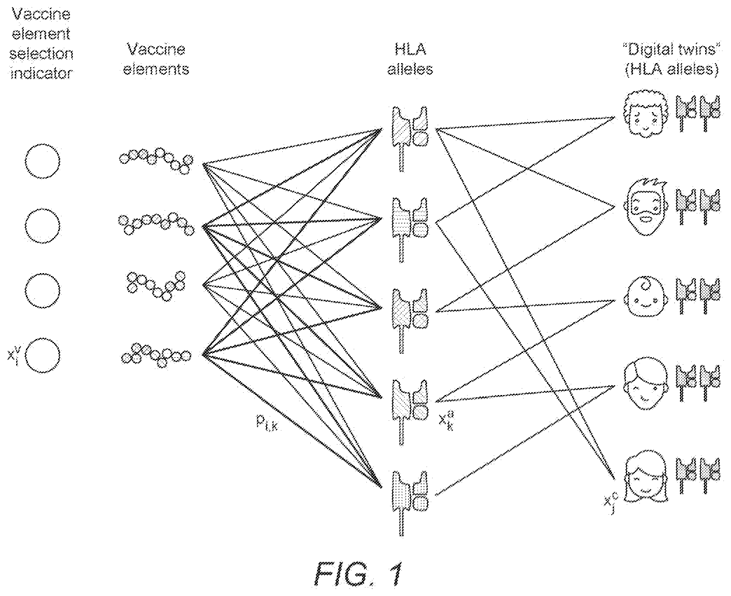

In this implementation this minimax problem is approached as a type of network flow problem, with one set of nodes corresponding to vaccine elements, one set corresponding to components of an immune profile (e.g. HLA alleles), and one set corresponding to citizens. The goal is to select the set of vaccine elements such that the likelihood of no response is minimized for each citizen. FIG. 1 gives an overview of the problem setting.

### VACCINE DESIGN PROCESS

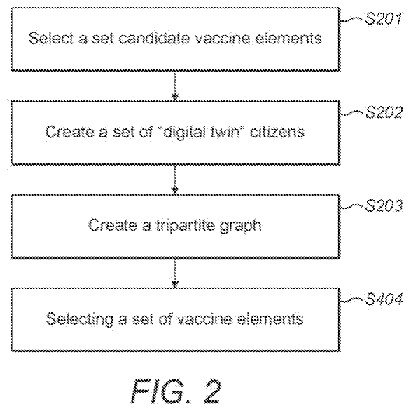

Concretely, we approach the vaccine design process in four steps, as shown in FIG. 2:
1. Select a set of candidate vaccine elements for inclusion in the vaccine (S201).
2. Create a set of “digital twin” citizens for a population of interest, where a digital twin is a representative immune profile (e.g. a set of HLA alleles, S202).
3. Create a tripartite graph in which the nodes correspond to vaccine elements, components of the immune profile (e.g. HLA alleles), and citizens; edges correspond to relevant biological terms described below (S203).
4. Select a set of vaccine elements (respecting a given budget) such that the likelihood that each citizen has a positive response is maximized (or, equivalently, that the log likelihood of no response for each citizen is minimized, S204).

We now describe each of these steps in detail.

### Step 1: Select a Set of Candidate Vaccine Elements

Some of these candidate vaccine elements will be selected for inclusion in a vaccine. Four examples of vaccine elements are: (1) short peptide sequences, such as 9-mer amino acid sequences; (2) long peptide sequences, such as 27-mer amino acid sequence which may be based on a short peptide sequence and include flanking regions; (3) longer amino acid sequences which may include multiple short peptide sequences as well as the intervening, naturally-occurring sequence; and (4) entire protein sequences.

Each vaccine element vᵢ is associated with a cost cᵢ^{v}, while a total budget b is available for including elements in the vaccine. The description of the budget and costs depend on the vaccine platform.

Some vaccine platforms are mainly restricted to a fixed number of vaccine elements; in this case, each cost cᵢ^{v} will be 1, and the budget will indicate the total number of elements which can be included.

Some other vaccine platforms are restricted to a maximum length of included elements. In this case, each cost cᵢ^{v} will be the length of the vaccine element, and the budget will indicate the maximum length of elements which can be included.

### Step 2: Create a Set of “Digital Twin” Citizens

Our approach is based on simulating a set of “digital twin” citizens. In this example implementation, there is a focus on vaccine elements whose effects are determined, in part, by the HLAs of each citizen. Thus, each digital twin may corresponds to a set of HLA alleles (or an immune profile as described further below).

It is known that citizens from different regions of the world tend to have different sets of HLA alleles; further, some combinations of HLA alleles are more common than others (Cao, K.; Jill Hollenbach; Shi, X.; Shi, W.; Chopek, M. & Fernández-Vińa, M. A. Analysis of the frequencies of HLA-A, B, and C alleles and haplotypes in the five major ethnic groups of the United States reveals high levels of diversity in these loci and contrasting distribution patterns in these populations. Human Immunology, 2001, 62, 1009-1030). In certain implementations full HLA genotypes from actual citizens can be used to accurately model these relationships, the genotypes available from high-quality samples in the Allele Frequency Net Database (AFND, http://www.allelefrequencies.net/).

### Creating a Distribution Over Genotypes for Each Region:

In particular, AFND assigns each sample to a region based on where the sample came from (e.g., “Europe” or “Sub-Saharan Africa”). In a first step, posterior distribution over genotypes in each region may be created based on the observations and an uninformative (Jeffreys) prior distribution.

Specifically, all genotypes observed at least once across all regions can be collected and an index g assigned to each genotype. The total number of unique genotypes may be called G. Second, a prior distribution over genotypes may be specified. In certain implementations, a symmetric Dirichlet distribution may be used with a concentration parameter of 0.5 because this distribution is uninformative in an information theoretic sense and does not reflect strong prior beliefs that any particular genotypes are more likely to appear in any specific region. For each region, a posterior distribution over genotypes is then calculated as a Dirichlet distribution as follows.

θ₁, . . . , θ_{G}|x₁, . . . , x_{G}˜Dirichlet(α₁+x₁, . . . , α_{G}+x_{G})

where α_{g} is the (prior) concentration parameter for the g^{th} genotype (always 0.5 here) and x_{g} is the number of times the g^{th} genotype was observed in the region.

This distribution can now be used to sample genotypes from a region using a two-step process.

θ₁, . . . , θ_{G}˜Dirichlet(α₁+x₁, . . . , α_{G}+x_{G})

y₁, . . . , y_{G}˜Multinomial(θ₁, . . . , θ_{G}; n)

where n is the desired number of genotypes to sample from the region, and y₁, . . . , y_{G} are the counts of each genotype in the sample.

### Creating a Set of “Digital Twin” Citizens:

The example implementation continues by creating a set of digital twin citizens using a two-step approach. The method is preferably given the population size p, as well as a distribution over regions. Concretely, the input is a Dirichlet distribution over the regions, as well as p (note that this Dirichlet is completely independent of those over genotypes discussed in the previous section).

The Dirichlet distribution over regions has one “concentration” parameter for each region; each parameter reflects the proportion of digital twins for the population which come from that region. As one example, the parameters could be based on the actual populations of each region (e.g., https://www.worldometers.info/world-population/population-by-region/). The Dirichlet parameters must be positive, but they do not need to sum to 1. A sample from a Dirichlet distribution is a categorical distribution. That is, a sample from this Dirichlet (plus the population size) gives a multinomial distribution. That distribution may then be sampled to find the number of citizens from each region. Mathematically, we have the following, two-step sampling process.

θ₁, . . . , θ_{R}˜Dirichlet(α₁, . . . , α_{R})

d₁, . . . , d_{R}˜Multinomial(θ₁, . . . , θ_{R}; p)

where R is the number of regions, p is the desired population size, d₁, . . . , d_{R} are the counts of digital twins from each region, and α₁, . . . , α_{R} are the Dirichlet concentration parameters (given by the user).

Second, the genotypes for each region are sampled using the posterior distributions over genotypes discussed above. The number of genotypes sampled for region r is given by dᵣ.

In sum, there are two Dirichlet distributions. One is over the immune profiles or HLA genotypes (and is based on the observed genotypes), while the second is over the regions (and in certain implementations may be given by a user when running the simulations).

Simulating the population is then two steps:
1. Select how many digital twins come from each region (using the second, user-defined Dirichlet).
2. Select the genotypes for each digital twin based on his or her region (using the first Dirichlet based on the observed data).

### Step 3: Create a Tripartite Graph

In this provided example, a tripartite graph may be created. The graph may be a representation of how the specific problem may be solved however it will of course be understood that the graph may not be created but may be merely representative. Thus, in the next step of the example implementation, use the vaccine elements and digital twins may be used to construct a tripartite graph that will form the basis of the optimization problem for vaccine design. The graph has three sets of nodes:
1. All candidate vaccine elements identified in Step 1
2. All components of the immune profile, for example all HLA alleles in all digital twin genotypes
3. All digital twins

The graph may also have two sets of weighted edges:
1. An edge from each vaccine element vᵢ to each component, e.g. HLA allele, aₖ. The weight of this edge is log P(R=−|vᵢ, aₖ), that is, the likelihood of no response for the component from that particular vaccine element Note, below an approach is described for calculating this value for short peptides. Further, below a specific approach is described where the component of the immune profile is not an HLA allele.
2. An edge from each component or allele to each citizen which has that allele in its genotype (or the component in its immune profile). The weight of these edges is typically 1.

As an intuition, we call the edges from a vaccine element to an allele (and, then, from the allele to each patient with that allele) as “active” when the vaccine element is selected. Then, the log likelihood of response for a citizen is the sum of all active incoming edges. That is, the flow from selected vaccine elements to the citizens gives the likelihood of no response for that citizen.

$${\sum }_{{v}_{i} ∈ V} {\sum }_{{a}_{k} ∈ A ( {c}_{j} )} log P ( R = - \vert {v}_{i} , {a}_{k} )$$

### Calculating the Likelihood of No Response for a Given Digital Twin and Vaccine Elements:

The following describes example approaches for calculating log P(R=−|vᵢ, aₖ) for three types of vaccine elements. The vaccine design approach is applicable for any approach which assigns a value for log P(R=−|vᵢ, aₖ).
1. Short peptide sequences. Most short peptide prediction engines compute some sort of a score that a peptide will result in some immune response (e.g., binding, presentation, cytokine release, etc.), and this score generally takes into account a specific HLA allele (Jensen, K. K.; Andreatta, M.; Marcatili, P.; Buus, S.; Greenbaum, J. A.; Yan, Z.; Sette, A.; Peters, B. & Nielsen, M. Improved methods for predicting peptide binding affinity to MHC class II molecules. Immunology, 2018, 154, 394-406). In some cases, this is already a probability, and in others, it can be converted into a probability using a transformation function, such as a logistic function. Examples will be described below of scores where the response is for components other than an HLA allele.
We note that typically in the art, the terms likelihood and probability are used interchangeably and they are used interchangeably herein.
Thus, the prediction engines give P(R=+|vᵢ, aₖ), where vᵢ is the peptide and aₖ is the allele. One can then take log P(R=−|vᵢ, aₖ)=log[1−P(R=+|vᵢ, aₖ)].
2. Long peptide sequences. Longer peptide sequences may include multiple short peptide sequences with different scores from the prediction engine. An example approach to calculate log P(R=−|vᵢ, aₖ), where v is the long peptide sequence, is to take the minimum (i.e., best) log P(R=−|p, aₖ), where p is any short peptide contained in vᵢ.
3. Longer amino acid sequences. Longer amino acid sequences may contain even more short peptide sequences, and the same approach used for long peptide sequences can be used here.

### Step 4: Selecting a Set of Vaccine Elements

Finally, the vaccine design problem can be posed as a type of network flow problem through the graph defined in Step 3. In particular, the minimization problem can be posed as an integer linear program (ILP); thus, it can be provably, optimally solved using known ILP solvers.

### Handling the Minimax Problem:

As previously described, a goal is to choose the set of vaccine elements which minimize the log likelihood of no response for each patient or individual.

The minimax problem simplifies as follows.

$${min}_{V} {max}_{c ∈ C} {\sum }_{{v}_{i} ∈ V} {\sum }_{{a}_{k} ∈ A ( {c}_{j} )} log P ( R = - \vert {v}_{i} , {a}_{k} )$$

Thus, the terms inside the summation are exactly those calculated in Step 3 as the weights on the edges in the graph.

Standard ILP solvers cannot directly solve this minimax problem; however, in an example implementation proposed the approach uses of a set of surrogate variables to address this problem. In particular, define xⱼ^{c} is defined to be the log likelihood of no response for citizen cⱼ. That is, xⱼ^{c}:=Σᵥ_{ᵢ}_{∈V}Σₐ_{ₖ}_{∈A(c}_{ⱼ}₎log P(R=−|vᵢ, aₖ). Further,

$$z := {max}_{{c}_{j} ∈ C} {x}_{j}^{c}$$

may be defined; that is, z is the maximum log likelihood that any citizen does not respond to the vaccine (or, alternatively, the minimum log likelihood that any citizen will respond to the vaccine). Finally, then, the aim is to minimize z.

### ILP Formulation:

An example ILP formulation consists of three types of variables:
xᵢ^{v}: one binary indicator variable for each vaccine element which indicates whether it is included in the vaccine for the given population. Typically vaccine elements may be indexed with i.
xⱼ^{c}: one continuous variable for each citizen in the population which gives the log likelihood of no response for that citizen. Typically citizens may be indexed with j.
xₖ^{a}: one continuous variable for each HLA allele which gives the log likelihood of no response for that allele. Typically alleles may be indexed with k.
z: one continuous variable which gives the maximum log likelihood that any citizen does not respond to the vaccine (a goal may be to minimize this value.)

Additionally, the ILP uses the following constants:
p_{i,k}: the log likelihood that vaccine element vᵢ does not cause a response for allele k.
cᵢ^{v}: the “cost” of vaccine element vᵢ.
b: the maximum cost of vaccine elements which can be selected.

Finally, the ILP uses the following constraints:
xₖ^{a}=Σᵢp_{i,k}·xᵢ^{v}: one constraint for each allele which gives the log likelihood that at least one selected peptide results in a positive response for that allele
xⱼ^{c}=Σₐ_{ₖ}_{∈A(c}_{ⱼ}₎xₖ^{a}: one constraint for each citizen which gives the log likelihood that at least one selected peptide results in a positive response for at least one allele for that citizen (that is, this is the likelihood of a positive response for this citizen.)
b≥Σᵢcᵢ^{v}·xᵢ^{v}: the vaccine elements we select cannot exceed the budget
z≥xⱼ^{c}: as discussed above, we use z as an approach to solve the minimax problem. These constraints imply that z is the minimum log likelihood that any individual patient will respond to the vaccine.

The objective of the ILP is to minimize z.

The setting of the binary xᵢ^{v} variables corresponds to the optimal choice of vaccine elements for the given population.

Relationships to Max-Flow and Other Problems with Provably Efficient Solutions:

It is proposed that there is a relationship to max-flow and other problems with provable efficient solutions. This is highly-related to a number of efficiently solvable network flow problems. The proposed optimisation problem is essentially a min-flow problem with multiple sinks, where each citizen is a sink; however, the aim is to minimize the flow to each individual sink rather than the flow to all sinks. In particular, rather than the “sum” operator typically used to transform multiple sink flow problems into a single-sink problem, there is a need for a (non-linear) “min” operator. Thus, efficient min-flow formulations are not applicable in this setting.

The objective of the ILP remains to minimize z.

The setting of the binary xᵢ^{v} variables again corresponds to the optimal choice of vaccine elements for the given population.

### Immune Profiles:

As noted above as well as representing a set of HLA alleles for a population, the concept may also be used to represent an immune profile for a population, where the immune profile may optionally include the set HLA alleles as well as the other components or simply a set of other components that represent how the vaccine elements will respond in that representative population.

The following sets out examples of how the implementations set out above, which are typically tailored for, and explained in the context of, a set of HLA alleles.

In these example, the various other immune profile components may also be represented as central nodes in the graph. In an implementation, only discretized versions of each variable may be considered. For example, where the component represents “tumor infiltrating lymphocytes (TILs) presence=high” or “CTLA4 presence=low” rather than “TILs=73.8”). Likewise, human papillomavirus (HPV) can be considered represented as a discrete, binary variable (“HPV=false”). Thus, these can still be sampled using the Dirichlet distributions already used to sample the HLAs for each immune profile.

It was noted above that there the central nodes represent other components to HLA alleles, a score or a measure of the immune response (used as the edge of the graph) may be determined differently. In a specific implementation, the immune response values can be calculated for each of the above markers by extracting univariate response statistics for previous literature. This value may still be considered the log likelihood of no response. For example, let's say that published statistics show that 52 patients have “High” TIL presence, while 110 have “Low” TIL presence; this allows for construction of a distribution for TIL presence. Thus, each digital twin or representative immune profile for the population (i.e. the right hand node of the graph) will have a value for each of these profile elements in addition to the HLAs.

If for example the probability of response is 80% for the “High” and (approximately) 45% for the “Low” group, then these numbers can be used to give the immune response values for TIL presence. A similar approach can be used for all of the other elements of the immune profile.

In constructing the graph, each immune profile element and value (e.g., “TILs presence=High” or “CTLA4 presence=Low”) may be represented as a centre node; each of these nodes is connected to the appropriate digital twin nodes (the same as with the HLAs).

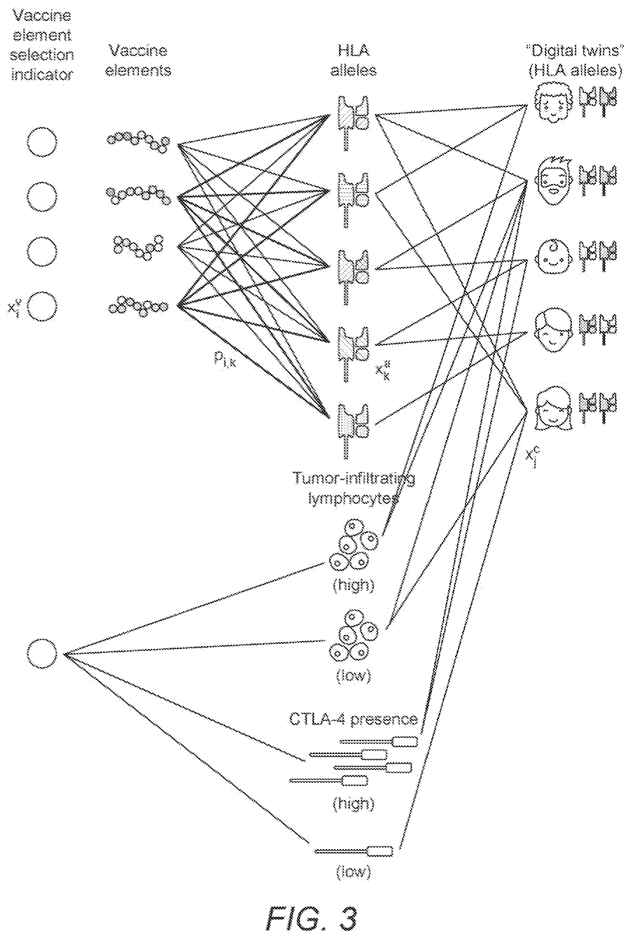

In certain example implementations a new node may be added to the first set of nodes in the graph (i.e. the candidate amino acid sequences); all of these immune profile element nodes are connected to this node, and the weight is the immune response value calculated, as described above. Such a graph is shown in FIG. 3.

In practice, this graph construction implies that the selected amino acid sequences do not “affect” the immune profile elements. Nevertheless, this construction will encourage the vaccine design to help digital twins with poor prognosis (e.g., “TILs presence=Low”).

### Creating a Vaccine for a Specific Vaccine Platform:

The choice of the vaccine delivery platform is potentially important for determining the budget for how many vaccine elements can be chosen, the costs of each vaccine element, and, eventually, how the actual vaccines are created based on the vaccine elements. The following provides two concrete examples of a vaccine platform and the resulting budget, costs, and use of the selected elements.

A first example uses the HCVp6-MAP vaccine. This “multiple antigenic peptide” (MAP) vaccine is designed as a preventative vaccine for Hepatitis C Virus (HCV). In the original study, the authors select short peptides as the vaccine elements based on several criteria. After selection, the short peptides were synthesized using the 9-fluorenylmethoxy carbonyl method. The peptides were then dissolved in DMSO at a concentration of 10 μg/μL and stored at −20° C. Just before immunization, peptides were diluted to the desired dose concentration (e.g., 800 ng per peptide in μL of DMSO) and were kept at 4° C. The vaccine was then administered subcutaneously (Dawood, R. M.; Moustafa, R. I.; Abdelhafez, T. H.; El-Shenawy, R.; El-Abd, Y.; Bader El Din, N. G.; Dubuisson, J. & El Awady, M. K. A multiepitope peptide vaccine against HCV stimulates neutralizing humoral and persistent cellular responses in mice. BMC Infectious Diseases, 2019, 19).

Mapping the HCVp6-MAP vaccine onto the present vaccine design problem, each vaccine element is a short peptide, the total budget is 6, and the cost of each vaccine element is 1. The selected vaccine elements can be processed as described to manufacture the vaccine.

As a second example, we consider the chimeric Hepatitis B surface antigen (HBsAg) DNA vaccine (Woo, W.-P.; Doan, T.; Herd, K. A.; Netter, H.-J. & Tindle, R. W. Hepatitis B Surface Antigen Vector Delivers Protective Cytotoxic T-Lymphocyte Responses to Disease-Relevant Foreign Epitopes. Journal of Virology, 2006, 80, 3975-3984). Roughly, this vaccine platform replaces two peptide sequences in the HBsAg small envelope protein with vaccine elements. In order to ensure immunogenicity of the molecule, the total length of the replacement vaccine elements must be approximately 36 amino acids (Trovato, M. & De Berardinis, P. Novel antigen delivery systems. World Journal of Virology, 2015, 4, 156-168). For the present vaccine design formulation, the total budget is 36, and the cost of each vaccine element is the length (in amino acids) of that element. Further details are known in the art on the technical details on synthesizing the DNA-based vaccine once the vaccine elements are selected (Woo, W.-P.; Doan, T.; Herd, K. A.; Netter, H.-J. & Tindle, R. W. Hepatitis B Surface Antigen Vector Delivers Protective Cytotoxic T-Lymphocyte Responses to Disease-Relevant Foreign Epitopes. Journal of Virology, 2006, 80, 3975-3984).

In summary, the proposed approach, according to an embodiment, includes the following steps:
1. Select a set of candidate vaccine elements for inclusion in the vaccine.
2. Create a set of “digital twin” citizens for a population of interest, where a digital twin is a set of HLA alleles or an immune profile.
3. Create a tripartite graph in which the nodes correspond to vaccine elements, HLA alleles (or portions of an immune profile), and citizens; edges correspond to relevant biological terms described below.
4. Select a set of vaccine elements (respecting a given budget) such that the likelihood that each citizen has a positive response is maximized (or, equivalently, that the log likelihood of no response for each citizen is minimized).

Implementations of embodiments of the present invention have particular utility to select peptide sequences for use in a prophylactic vaccine against SARS-CoV-2.

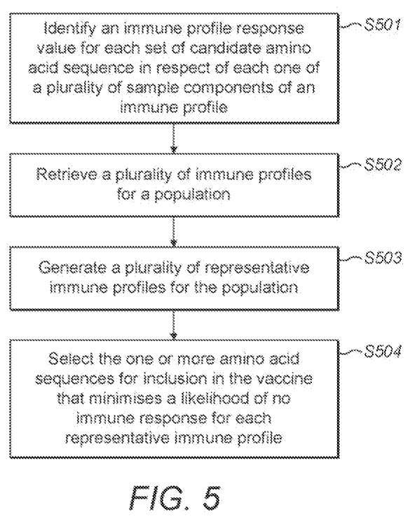

With reference to FIG. 5, a specific example implementation will now be described. At step S501, the method identifies an immune profile response value for each candidate amino acid sequence in respect of each one of a plurality of sample components of an immune profile. The immune profile response value represents whether the candidate amino acid sequence results in an immune response for the sample component of an immune profile. At step S502, the method retrieves a plurality of immune profiles for a population. At step S503, the method generates a plurality of representative immune profiles for the population. The representative immune profiles overlap with the sample components of an immune profiles. Finally, at step S504, the method selects the one or more amino acid sequences for inclusion in the vaccine that minimises a likelihood of no immune response for each representative immune profile, based on the immune profile response values.

### Example:

The following provides an implemented example of the above processes and concepts.

### A Graph-Based “Digital Twin” Optimization Prioritizes Epitope Hotspots to Select Universal Blueprints for Vaccine Design:

In order to develop a blueprint for viable universal vaccine against SARS-CoV-2, it is necessary to 1) cover with fidelity a broad proportion of the human population, and 2) prioritize the selection to even fewer regions (the exact number may depend on the size of the bin and the vaccine platform under consideration). Consequently, we need to identify the optimal constellation of hotspots, or relevant viral segments, that can provide broad coverage in the human population with a limited and targeted vaccine “payload”. In order to achieve this aim, we developed and applied a “digital twin” method, which models the specific HLA background of different geographical populations. A graph-based mathematical optimization approach is then used to select the optimal combination of immunogenic epitope hotspots which will induce immunity in the broad human population. Example output from an analysis are shown in FIG. 3. The output shows identified a subset of hotspots that may be combined to stimulate a robust immune response in a global population.

### Graph-Based Optimization in Digital Twin Simulations of the Epitope Hotspots:

We consider a population as a set C of “digital twin” citizens c, and a vaccine as a set V of vaccine elements v. We denote the likelihood that all citizens have a positive response to a vaccine as P(R=+|C, V). Our goal is to design a vaccine, that is, select a set of vaccine elements, to maximize this probability:

$${max}_{V} P ( R = + \vert V , C )$$

In this setting, maximizing the probability of positive response is the same as minimizing the probability of no response. Thus, we approach vaccine design by minimizing the probability of no response for the citizen who has the highest probability of no response P(R=−|V, cⱼ):

$${max}_{V} P ( R = + \vert V , C ) := {min}_{V} {max}_{{c}_{j} ∈ C} \{ P ( R = - \vert V , {c}_{j} ) \}$$

We consider that a vaccine causes a response if at least one of its elements causes a positive response. That is, the probability of no response is the joint likelihood that all elements fail. For a particular citizen cⱼ, this probability is given as follows.

$$P ( R = - \vert V , {c}_{j} ) = {\prod }_{{v}_{i} ∈ V} P ( R = - \vert v , {c}_{j} , V )$$

The original optimization problem can then be expressed as:

$${min}_{V} {max}_{{c}_{j} ∈ C} {\prod }_{{v}_{i} ∈ V} P ( R = - \vert {v}_{i} , {c}_{j} , V )$$

Since the logarithm function is monotonic, the value of V which minimizes the logarithm of the function also minimizes the original function.

$${min}_{V} {max}_{{c}_{j} ∈ C} {\prod }_{{v}_{i} ∈ V} log P ( R = - \vert {v}_{i} , {c}_{j} , V )$$

Further, we consider each citizen as a set of HLA alleles, and we assume that each vaccine element vᵢ may result in a response on each allele independently; we refer to the alleles for citizen cⱼ as A(cⱼ). Thus, our final objective is as follows.

$${min}_{V} {max}_{c ∈ C} {\sum }_{{v}_{i} ∈ V} {\sum }_{{a}_{k} ∈ A ( c )} log P ( R = - \vert {v}_{i} , k , V )$$

We approach this minimax problem as a type of network flow problem, with one set of nodes corresponding to vaccine elements, one set corresponding to HLA alleles, and one set corresponding to citizens. The goal is to select the set of vaccine elements such that the likelihood of no response is minimized for each citizen.

### Vaccine Design Process:

Concretely, we approach the vaccine design process in four steps:
1. Select a set of candidate vaccine elements for inclusion in the vaccine.
2. Create a set of “digital twin” citizens for a population of interest, where a digital twin is a set of HLA alleles.
3. Create a tripartite graph in which the nodes correspond to vaccine elements, HLA alleles, and citizens; edges correspond to relevant biological terms described below.
4. Select a set of vaccine elements (respecting a given budget) such that the likelihood that each citizen has a positive response is maximized (or, equivalently, that the log likelihood of no response for each citizen is minimized).

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A computational intelligence-implemented method of selecting a set of candidate vaccine elements for inclusion in a vaccine, the method comprising:
creating a set of digital twin citizens for a population of interest, where a digital twin is a set of human leukocyte antigen (HLA) alleles or an immune profile;
creating a tripartite graph in which nodes correspond to the vaccine elements, the HLA alleles, and the citizens; and
selecting (i) a first set of vaccine elements such that a likelihood that each citizen has a positive response is maximized or (ii) a second set of vaccine elements such that a likelihood of no response for each citizen is minimized.

2. The computational intelligence-implemented method of claim 1, wherein the method comprises:
identifying an immune profile response value for each vaccine element with respect to each one of sample components of an immune profile, wherein the immune profile response value represents whether the respective vaccine element results in an immune response for the sample components of the immune profile;
retrieving a plurality of immune profiles for the population; and
generating a plurality of representative immune profiles for the population, wherein each of the plurality of immune profiles overlaps with the sample components of the immune profiles.

3. The computational intelligence-implemented method of claim 2, wherein the method comprises:
creating a first distribution over the plurality of immune profiles; and
sampling the first distribution to create the plurality of representative immune profiles.

4. The computational intelligence-implemented method of claim 3, wherein the first distribution is a distribution over the plurality of immune profiles for each region of the population.

5. The computational intelligence-implemented method of claim 4, wherein the first distribution is a posterior distribution over genotypes in each region based on a prior distribution and observed genotypes from the plurality of immune profiles in each region of the population.

6. The computational intelligence-implemented method of claim 5,
wherein the first distribution is a symmetric Dirichlet distribution,
wherein the method comprises:
collecting all genotypes observed at least once across all regions; and
sampling a desired number of genotypes from each region based on counts of each genotype in a sample.

7. The computational intelligence-implemented method of claim 3, wherein the method comprises:
simulating a digital population based on the retrieved plurality of immune profiles for the population; and
creating a first distribution based on the simulated population such that the sampling is performed on the distribution of the simulated population.

8. The computational intelligence-implemented method of claim 7, wherein the method comprises:
defining a population size; and
creating a second distribution over the regions.

9. The computational intelligence-implemented method of claim 8, wherein the second distribution is a Dirichlet distribution.

10. The computational intelligence-implemented method of claim 2, wherein the representative immune profiles are generated such the representative immune profiles maximize coverage of combinations of immune profiles in the population.

11. The computational intelligence-implemented method of claim 2, wherein the method comprises:
applying a mathematical optimization algorithm to minimize a maximum likelihood of no immune response for each of the representative immune profiles.

12. The computational intelligence-implemented method of claim 11,
wherein the immune profile comprises a set of HLA alleles, and
wherein variables of the mathematical optimization algorithm comprise:
(a) a binary indicator variable for each vaccine element which indicates whether the vaccine element is included in a vaccine;
(b) a continuous variable for each representative immune profile which gives a log likelihood of no immune response;
(c) a continuous variable for each sample component of an immune profile which gives a log likelihood of no response; and
(d) a continuous variable which gives a maximum log likelihood that any representative immune profile does not respond to the selected one or more vaccine elements,
wherein the mathematical optimization algorithm minimizes the continuous variable which gives a maximum log likelihood that any representative immune profile does not respond to the selected one or more vaccine elements.

13. The computational intelligence-implemented method of claim 11, wherein the mathematical optimization algorithm is a mixed integer linear program.

14. The computational intelligence-implemented method of claim 2, wherein the method comprises:
assigning a cost to each vaccine element; and
selecting is constrained based on the cost assigned to each vaccine element, such that the selected one or more vaccine elements have a total cost below a predetermined threshold budget.

15. The computational intelligence-implemented method of claim 2, wherein the method comprises:
selecting is constrained based on a maximum amount of vaccine elements allowed in a vaccine delivery platform.

16. The computational intelligence-implemented method of claim 2,
wherein the method comprises:
creating a tripartite graph,
wherein: a first set of nodes corresponds to the vaccine elements; a second set of nodes corresponds to the sample components of an immune profile; and a third set of nodes corresponds to the representative immune profiles for the population, and
wherein: weights of edges between the first set of nodes and the second set of nodes are the immune response values; and weights of edges between the second set of nodes and the third set of nodes represent correspondence between the sample components of an immune profile and each representative immune profile.

17. The computational intelligence-implemented method of claim 2, wherein the immune response value is a log likelihood value based on amino acid subsequences of the candidate vaccine element.

18. The computer implemented method of claim 2, wherein the method comprises:
selecting a best likelihood value as the immune response value from a likelihood value for each amino-acid subsequence.

19. The computational intelligence-implemented method of claim 2, wherein the one or more vaccine elements are comprised in one or more proteins of a coronavirus, preferably the SARS-CoV-2 virus.

20. The computational intelligence-implemented method of claim 2, wherein the representative immune profile comprises one or more selected from a group comprising: a set of HLA alleles; presence of tumor infiltrating lymphocytes; presence of immune checkpoint markers; presence of hypoxia markers; presence of chemokine receptors; and previous infection by human papillomavirus.

21. The computational intelligence-implemented method of claim 2, wherein the method comprises:
selecting the one or more vaccine elements for inclusion in the vaccine based on a correspondence between the sample components of an immune profile and the representative immune profiles.

22. A non-transitory computer readable medium having computer executable instructions stored thereon for implementing a method of selecting a set of candidate vaccine elements for inclusion in a vaccine, the method comprising:
creating a set of digital twin citizens for a population of interest, where a digital twin is a set of human leukocyte antigen (HLA) alleles or an immune profile;
creating a tripartite graph in which nodes correspond to the vaccine elements, the HLA alleles, and the citizens; and
selecting (i) a first set of vaccine elements such that a likelihood that each citizen has a positive response is maximized or (ii) a second set of vaccine elements such that a likelihood of no response for each citizen is minimized.
